(19) United States
(12) Patent Application Publication
(10) Pub. No.: US 2023/0210708 A1
(43) Pub. Date: Jul. 6, 2023

(54) MECHANICAL FOOT

(71) Applicant: ANGELEXO SCIENTIFIC CO., LTD, Xiaoshan District Hangzhou (CN)

(72) Inventors: Yanjun FAN, Xiaoshan District Hangzhou (CN); Xiaoze WANG, Xiaoshan District Hangzhou (CN); Sen YANG, Xiaoshan District Hangzhou (CN); Xuehua CHEN, Xiaoshan District Hangzhou (CN); Luya LI, Xiaoshan District Hangzhou (CN)

(73) Assignee: ANGELEXO SCIENTIFIC CO., LTD, Xiaoshan District Hangzhou (CN)

(21) Appl. No.: 18/091,747

(22) Filed: Dec. 30, 2022

Related U.S. Application Data
(63) Continuation-in-part of application No. PCT/CN2022/126060, filed on Oct. 19, 2022.

(30) Foreign Application Priority Data
Oct. 19, 2021 (CN) 202111214229.1

(51) Int. Cl. A61H 3/00; A61H 1/02

(52) U.S. Cl. CPC A61H 3/00; A61H 1/0266; A61H 2003/007; A61H 2201/1642; A61H 2201/165; A61H 2201/1261

(57) ABSTRACT

A mechanical foot includes a mechanical ankle joint including a leg portion configured to be fixed to a patient's leg, a foot portion configured to be fixed to the patient's foot, and a revolute. The leg portion and the foot portion are connected through the revolute in a manner of being rotatable relative to each other. The revolute includes a foot connecting portion connected to the foot portion and a leg connecting portion connected to the leg portion, and the foot connecting portion and the leg connecting portion are connected in a manner of being rotatable relative to each other. The revolute further includes a first elastic element having one end connected to the foot connecting portion, and the other end connected to the leg connecting portion, so that a force maintaining the patient's foot substantially horizontal is provided through the revolute at a position of the mechanical ankle joint.

### CROSS-REFERENCE TO RELATED APPLICATION

The present disclosure is a continuation-in-part application of International application No. PCT/CN2022/126060, filed on Oct. 19, 2022, which claims the priority to the Chinese patent application with the filing No. 202111214229.1, filed on Oct. 19, 2021 with the Chinese Patent Office, and entitled “Mechanical Foot”, the contents of which are incorporated by reference herein in entirety.

### TECHNICAL FIELD

The present disclosure relates to the technical field of medical devices, in particular to a mechanical foot.

### BACKGROUND ART

For patients with lower limb weakness, such as post-stroke patients, movement of their diseased limb is uncontrollable or difficult to control. Due to these reasons, when these patients' leg is lifted, their foot portion always naturally drops around the ankle joint under the action of its own gravity. If the problem of foot drop is not solved, these patients are likely to drag their foot on the ground while walking, or trip, tumble, therefore, it is quite important to prevent foot drop.

Chinese patent with the grant announcement number CN105722490B discloses a device capable of reliably lifting a person's toes during swing of lower limbs, wherein an ankle strap thereof is provided with a retractable elastic element, the retractable elastic element is connected to a foot by a cable, and the ankle is fixed in dorsiflexion by the cable during the swing of lower limb.

This approach does can solve the problem of foot drop to some extent, but it also has a number of problems: 1. the cable is directly connected to the foot, and in the working process, the force generated by dorsiflexion is concentrated at one position of the ankle, it is easy to cause discomfort at the ankle portion of the patient; 2. a pulling force of the cable is controlled by the retractable elastic element, and how to control the elastic force of the retractable elastic element is quite difficult, which is easy to cause different degrees of dorsiflexion during each time of lower limb swing, resulting in discomfort of the patient; and 3. as the cable fixed on the foot is always exposed outside, the cable is easy to be caught by other objects in the walking process of the patient, and the patient is caused to fall down.

### SUMMARY

The present disclosure aims at providing a mechanical foot, so as to solve the problem that the patients easily fall down in the background art.

In order to achieve the above objective, the technical solution used in the present disclosure is a mechanical foot.

The mechanical foot includes a mechanical ankle joint, wherein the mechanical ankle joint includes a leg portion configured to be fixed to a patient's leg, a foot portion configured to be fixed to the patient's foot, and revolute(s) (revolving pair(s)), the leg portion and the foot portion are connected through the revolute in a manner of being rotatable relative to each other, and wherein the revolute includes a foot connecting portion and a leg connecting portion, the foot connecting portion is connected to the foot portion, the leg connecting portion is connected to the leg portion, and the foot connecting portion and the leg connecting portion are connected with each other in a manner of being rotatable relative to each other.

Optionally, the revolute further includes a first elastic element, one end of the first elastic element is connected to the foot connecting portion, and the other end of the first elastic element is connected to the leg connecting portion, so that a force maintaining the patient's foot substantially horizontal is provided through the revolute at a position of the mechanical ankle joint.

Optionally, the leg connecting portion is provided with a hole, the foot connecting portion is provided with a shaft, and the shaft is configured in such a way of being provided in the hole in a rotatable manner.

Optionally, the leg connecting portion is provided with a first connection supporting point, the foot connecting portion is provided with a second connection supporting point, and the first connection supporting point is connected with the second connection supporting point through the first elastic element.

Optionally, the foot connecting portion is provided with side walls, and when the leg connecting portion and the foot connecting portion rotate relative to each other, the side walls limit a movement position of the first connection supporting point.

Optionally, the leg connecting portion is provided thereon with a connecting rod, and the first connection supporting point is arranged at an end of the connecting rod away from the leg portion.

Optionally, the end of the first elastic element connected to the first connection supporting point is configured to rotate freely around a central axis of the first connection supporting point, and the other end of the first elastic element connected to the second connection supporting point is configured to rotate freely around a central axis of the second connection supporting point.

Optionally, the second connection supporting point is provided at a bottom position of the foot connecting portion close to a side wall of the side walls.

Optionally, the mechanical ankle joint further includes a cover plate, and an edge of the cover plate is of a shape matched with shapes of the side walls of the foot connecting portion.

Optionally, the foot portion of the mechanical foot includes a rear portion and a forefoot portion, the rear portion and the forefoot portion constitute a bottom portion attached to the patient's foot, the foot connecting portion is connected to the bottom portion, and the rear portion is movably connected to the forefoot portion.

Optionally, the foot portion of the mechanical foot includes a forefoot rear-side portion and a forefoot portion, the forefoot rear-side portion and the forefoot portion are movably connected with each other, the forefoot rear-side portion and the forefoot portion constitute a bottom portion attached to the patient's foot, the foot connecting portion is connected to the bottom portion, and a damping structure is provided at a junction between the forefoot rear-side portion and the forefoot portion, so as to make the forefoot rear-side portion angularly changed relative to the forefoot portion.

Optionally, the damping structure includes a damping rotating shaft, the forefoot rear-side portion is provided thereon with a first mounting station, the forefoot portion is provided thereon with a second mounting station, and the first mounting station and the second mounting station are each provided with a first connecting hole for allowing the damping rotating shaft to be mounted.

Optionally, a limiting structure is provided at a connecting position of the forefoot rear-side portion and the forefoot portion (i.e. at a portion where the forefoot rear-side portion is connected with the forefoot portion), so as to avoid excessive rotation of the damping rotating shaft.

Optionally, the forefoot portion is provided with a first groove configured to face the first mounting station, and the first mounting station is configured in such a manner that when the forefoot portion rotates to an extreme position, the first mounting station partially extends into the first groove, so as to abut against the first groove.

Optionally, a second groove is provided on a side portion of the first mounting station facing the first groove, so that the forefoot portion can be inclined downwards to meet plantarflexion requirement of the patient.

Optionally, the forefoot portion and the forefoot rear-side portion are connected in a rotatable manner through an elastic rotating shaft, a second elastic element is provided inside the elastic rotating shaft, and the second elastic element is configured in such a manner that when the mechanical foot is not worn or when the patient wears the mechanical foot and lifts the foot up, the forefoot portion forms an angle with the forefoot rear-side portion under action of a biasing force of the second elastic element; and that when the patient wears the mechanical foot and steps on the ground with the foot, a force applied by the patient resists the biasing force applied by the second elastic element, so that the forefoot portion is flush with the forefoot rear-side portion.

Optionally, the leg portion and the bottom portion have an angle of 92°-95°.

To sum up, the beneficial effects of the present disclosure are as follows: in the present disclosure, the diseased forefoot of the patient can be lifted up during the walking of the patient, so as to prevent foot dragging and some injuries caused by tripping or tumbling related to foot dragging; the acting force generated by the second elastic element acts on the patient's forefoot, and will not generate an over-large force on the ankle joint of the patient to cause discomfort, thus solving the problem of discomfort caused by focusing all forces on the ankle joint in the background art; the elastic force of the second elastic element does not need to be controlled, they are determined by the weight of the foot portion of the patient, and the elastic force provided is matched with the weight of the corresponding portion, so that the forefoot upturning degree is the same during each time of lower limb swinging, and the discomfort of the patient is greatly reduced; and in addition, the technical solution of the present disclosure replaces the technical solution of rope drive in the prior art, and greatly reduces the possibility that the patient is tripped over.

### BRIEF DESCRIPTION OF DRAWINGS

In order to more clearly illustrate technical solutions in the embodiments of the present disclosure, drawings which need to be used in the description of the embodiments or the prior art will be introduced briefly below. Apparently, the drawings in the description below merely show an embodiment of the present disclosure. Those ordinarily skilled in the art still could obtain other relevant drawings in light of these drawings, without using creative effort.

FIG. 1 is an overall structural schematic diagram of a mechanical foot of the present disclosure in one direction;

FIG. 2 is an overall structural schematic diagram of the mechanical foot of the present disclosure in another direction;

FIG. 3 is a partial enlarged schematic diagram of an area “A” in FIG. 1;

FIG. 4 is an exploded schematic diagram of a layout structure of the mechanical foot in FIG. 1;

FIG. 5 is a partial enlarged schematic diagram of an area “B” in FIG. 4;

FIG. 6 is a further exploded schematic diagram of the mechanical foot in FIG. 4;

FIG. 7 is a structural schematic diagram of a foot connecting portion;

FIG. 8 is a left view of the mechanical foot with a cover plate being omitted;

FIG. 9 is a front view of the mechanical foot;

FIGS. 10A-D show schematic diagrams when a patient walks with a leg stepped on a second portion and a third portion; and

FIG. 11 is a partial enlarged schematic diagram of an area “C” in FIG. 8.

### DETAILED DESCRIPTION OF EMBODIMENTS

In order to make the technical problems to be solved by the present disclosure, technical solutions, and beneficial effects to be more clearly understood, the embodiments of the present disclosure will be further described in detail below in combination with drawings. It should be noted that the embodiments are only a detailed description of the present disclosure, and should not be regarded as limitation to the present disclosure. All the features disclosed in the embodiments of the present disclosure, or all the steps in the disclosed method or process, except for mutually exclusive features and/or steps, can be combined in any way.

In optional Embodiment 1, a mechanical ankle joint is provided, wherein the ankle joint includes a leg portion and a foot portion, the leg portion is configured to be fixed to a patient's leg, the foot portion is configured to be fixed to the patient's foot, and the two portions can be movably connected to each other, so as to facilitate people having lost the walking function in walking or performing rehabilitation training, which is particularly suitable for patients with hemiparalysis, or people with lower limb paralysis for performing lower limb rehabilitation training. The patient with lower limb paralysis or hemiparalysis herein mainly refers to patient whose lower limb is not controlled by cerebral nerves, but can recover nervous control through rehabilitation training. In some embodiments, the movable connection herein is a rotational connection, and the mechanical ankle joint includes a revolute, so that the leg portion and the foot portion are rotatable relative to each other. In some modes, the foot portion is fixed to the patient's foot by being connected to a rearfoot portion of the mechanical foot.

In some modes, such rotational connection is not a free rotational connection, but involves coordination with an elastic element, that is, the revolute has elastic resilience. Specifically, the rearfoot of the mechanical foot is fixedly connected with the rotatable foot portion, when the mechanical foot is worn, the patient's rearfoot is in contact with the rearfoot of the mechanical foot, the patient's forefoot is in contact with a forefoot of the mechanical foot, the rearfoot of the mechanical foot is connected with the foot portion, and the foot portion and the leg portion are in rotational connection with each other (ankle joint). Walking of a normal person is done by cooperation between the ankle joint and the sole (i.e. the sole of foot), for example, when walking forward, the sole (the sole being an area between heel and forefoot) is lifted from the ground by the shank, then or simultaneously, the heel is lifted from the ground, and then drives the forefoot to be lifted from the ground. In this process, the movement of the foot is accompanied by continuous adjustment of the ankle joint all the time, wherein at the moment that the forefoot is about to be lifted from the ground, the sole forms an angle with the ground, and the heel is vertex of this angle; subsequently, with the movement of the patient's leg, the patient's foot also performs a suspending movement; when the patient's foot is to land on the ground, according to different walking habits of the patients, for some people, it is the forefoot that lands first, and for some people, it is the heel that lands first, while regardless of the habits, there is always a moment when the foot is completely landed on the ground, so as to perform the next movement or walking. These movements of the sole, particularly starting and stepping, and angles of movement are closely adjusted through rotation of the ankle joint. However, for patients with paralysis, all of the movement of the ankle joint, the movement of the sole, including the angle between the sole and the ground, or the sequence and adjustment of the heel off the ground and in contact with the ground are nearly lost. Generally, when the mechanical foot is not worn, with the lifting of the leg, due to the gravity of the sole, the foot portion naturally drops around the ankle joint under the action of its own gravity, so as to drive the part of the heel to be in uncontrollable connection with the ankle joint, which is manifested as natural drop. The movement of the sole is mainly completed by the movement of the sole or the foot driven by the ankle joint.

However, after wearing the mechanical foot that has the mechanical ankle joint of the present embodiment, the revolute that has elastic resilience in the mechanical ankle joint can provide, from the ankle joint position, a force for lifting the patient's foot, in the process that the patient lifts the leg, and this force can keep the patient's foot portion as horizontal as possible for holding the patient's dropping foot, thus alleviating the condition of patient's foot drop. Different from “CN105722490B” (referred to as prior art hereinafter), in the present embodiment, the force for preventing the foot drop is transmitted from the ankle joint position of the patient, while in the prior art, the force for preventing the foot drop is transmitted from the sole position (especially the forefoot) of the patient, and the mechanical ankle joint of the present embodiment does not generate any pulling force on the sole position of the patient, and relatively has better comfort.

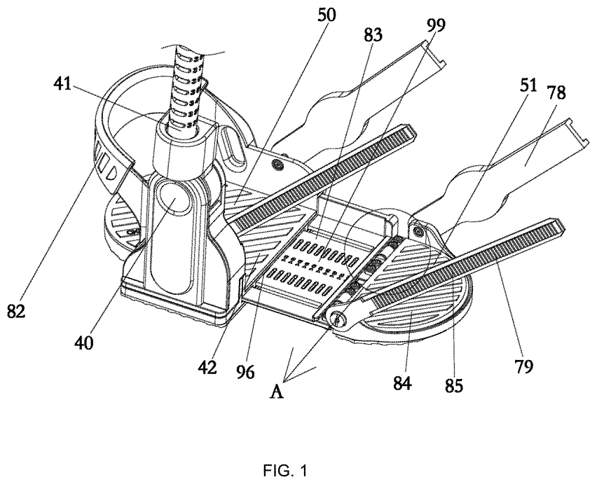

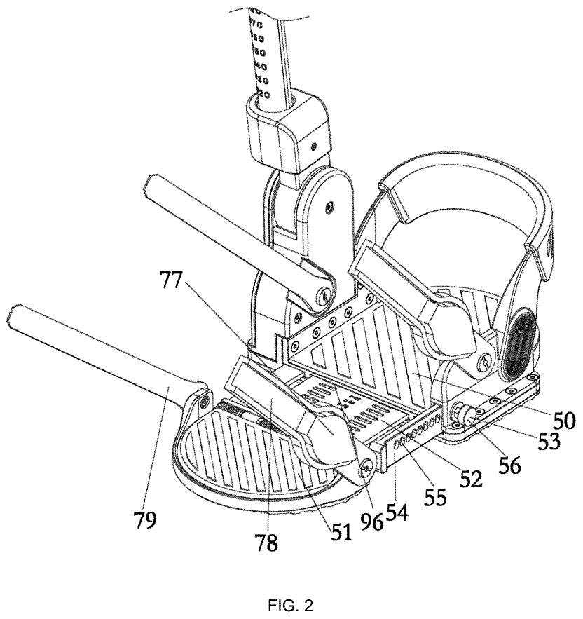

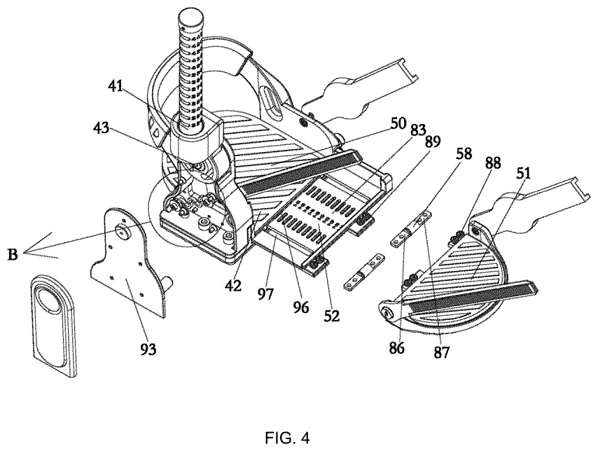

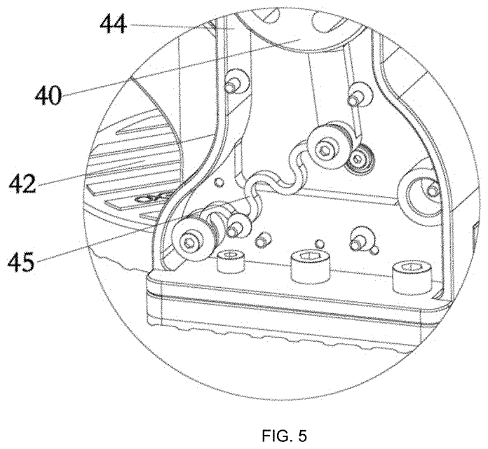

Specifically, referring to FIG. 1, the revolute is a second revolute 40, the leg portion is a portion numbered 41, and the foot portion is a portion numbered 42. Referring to FIG. 4 to FIG. 6, the mechanical ankle joint includes the leg portion 41 and the foot portion 42, the leg portion 41 and the foot portion 42 are connected in a rotating fit manner through the second revolute 40, and the second revolute 40 is located at the ankle joint position. The second revolute 40 includes a leg connecting portion 43 and a foot connecting portion 44, wherein the leg connecting portion 43 is connected to the leg portion 41, the foot connecting portion 44 is connected to the foot portion 42, the leg connecting portion 43 and the foot connecting portion 44 are nested together to form the rotatable second revolute 40. In the present embodiment, the leg connecting portion 43 is provided with a hole 46, the foot connecting portion 44 is provided with a shaft 47, and the shaft 47 of the foot connecting portion 44 can be inserted into the hole 46 on the leg connecting portion 43 to form the rotatable second revolute 40. Optionally, in order to reduce the wear of the shaft 47 and the hole 46 during rotation, a bearing or a bushing 48 is provided between the shaft 47 and the hole 46, and the bushing 48 may be a brass bushing or a lubrication-free bushing made of a special material, which can reduce the maintenance cost of the mechanical ankle joint.

A first elastic element is provided in the second revolute 40. The first elastic element makes the second revolute 40 have the capability of restoring to an initial state after rotation, and meanwhile also makes the movement speed of the second revolute 40 reduced during rotation, so that the movement of the ankle joint portion is as gentle as possible, and damages caused by too quick movement speed of the ankle joint are avoided. The first elastic element may be a coil spring, a tension spring, a spring, etc., and the first elastic element has one end connected to the foot connecting portion 44, and the other end connected to the leg connecting portion 43, such that when second revolute 40 rotates, i.e., when relative movement occurs between the foot connecting portion 44 and the leg connecting portion 43, the first elastic element will be stretched or compressed, and the first elastic element stores the elastic potential energy, so that the second revolute 40 has the capability of restoring to the initial state, meanwhile, during the rotation of the second revolute 40, due to the deformation of the first elastic element 40 itself, a force for blocking rotation is always provided to the position of the second revolute 40, so that the movement speed of the second revolute 40 can be reduced when rotating. Specifically, in the present embodiment, a rotating fit connecting portion of the leg connecting portion 43 has a circular profile, a first connection supporting point 90 is provided on an outer side of the outer profile thereof, and a second connection supporting point 91 is provided in the foot connecting portion 44, the first connection supporting point 90 is connected to the second connection supporting point 91 through a spring 45. With the rotation of the second revolute 40, a distance between the first connection supporting point 90 and the second connection supporting point 91 continuously keeps changing, and at this time, the length of the spring 45 also keeps changing continuously. In the present embodiment, the first elastic element provides a pushing force rather than a pulling force.

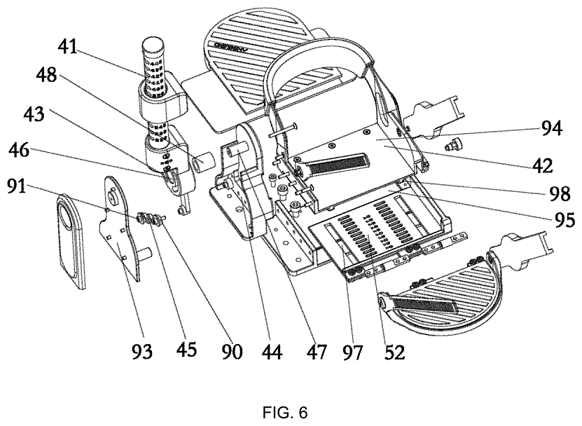

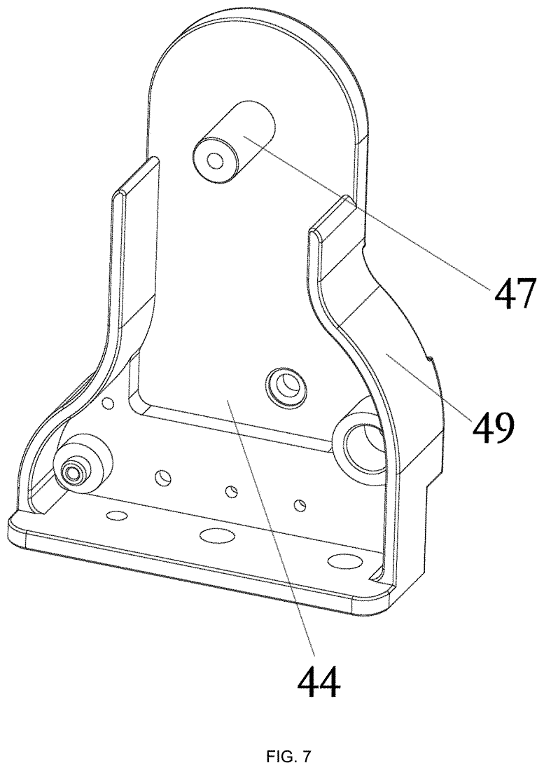

Preferably, in order to limit the maximum rotation angle of the mechanical ankle joint, preferably, referring to FIG. 6 and FIG. 7, the foot connecting portion 44 is provided with side walls 49, and when the leg connecting portion 43 and the foot connecting portion 44 rotate relative to each other, the first connection supporting point 90 on the leg connecting portion 43 abuts against the side walls 49 on the foot connecting portion 44, and when they abut against each other, it means that the second revolute 40 has moved to an extreme position, and a rotatable region of the second revolute 40 can protect the ankle of the patient. Optionally, two sides of the foot connecting portion 44 are each provided with the side wall 49 for limiting of two sides of the second revolute 40, so as to avoid excessive dorsiflexion or excessive dorsal extension of the patient's foot.

Preferably, the leg connecting portion 43 is provided thereon with a connecting rod 92, and the first connection supporting point 90 is arranged at an end of the connecting rod 92. The arrangement of the connecting rod 92 can adjust the position of the first connection supporting point 90. When the first connection supporting point 90 is provided at the end of the connecting rod 92, compared with the first connection supporting point 90 provided at an outer profile position of the leg connecting portion 43, as the position of the first connection supporting point 90 between the two side walls 49 is changed, correspondingly, a rotatable region of the second revolute 40 is also changed accordingly. By providing the connecting rod 92 of an appropriate length, the ankle has an appropriate maximum dorsiflexion angle and an appropriate maximum dorsal extension angle.

Preferably, two ends of the spring 45 are respectively sheathed on the first connection supporting point 90 and the second connection supporting point 91, the spring 45 is connected to contact positions of the first connection supporting point 90 and the second connection supporting point 91 in a sliding fit manner, so that the two ends of the spring 45 form two revolutes with the first connection supporting point 90 and the second connection supporting point 91, respectively. Such a design enables the spring 45 not to twist due to the rotation of the first connection supporting point 90 in the rotating process of the second revolute 40. If the two ends of the spring 45 are fixedly connected to the first connection supporting point 90 and the second connection supporting point 91, relative movement occurs between the first connection supporting point 90 and the second connection supporting point 91, along with the rotation of the first connection supporting point 90, and the spring at the positions where the two ends of the spring 45 are connected to the first connection supporting point 90 and the second connection supporting point 91 are prone to distortion and fracture. The sheathing mode solves this problem, and the spring 45 can always provide a pulling force in a direction of the connecting line of the first connection supporting point 90 and the second connection supporting point 91, to urge the second revolute 40 to return to an initial position.

Preferably, the second connection supporting point 91 is located at a bottom position of the foot connecting portion 44 close to the side wall 49 at one side, so that the spring 45 in the second revolute 40 has sufficient installation space and movement space, and is not easily interfered with other parts. At the same time, in the present embodiment, since the second connection supporting point 91 is located at the bottom position of the side wall 49 close to the heel side, the spring 45 in the second revolute 40 is enabled to be arranged in an inclined manner, and such inclined manner is that a lower end of the spring 45 is biased towards the heel position. The arrangement of this structure, in cooperation with the pushing force provided by the spring 45, enables the forefoot and the heel of the mechanical foot to be approximately horizontal; in some other embodiments, the second connection supporting point 91 is located at a bottom position of the side wall 49 close to the forefoot side, at this time, the spring 45 in the second revolute 40 is still arranged in an inclined manner, but the inclined manner is that the lower end of the spring 45 is inclined to the forefoot position, at this time, the spring 45 needs to provide a pulling force to make the forefoot and the heel of the mechanical foot approximately horizontal. According to different positions where the spring 45 is arranged, the form of the pulling force or the elastic force provided thereby is also different. Relatively speaking, the arrangement in which the second connection supporting point 91 is located at the bottom position of the side wall 49 close to the heel side is slightly superior to that in which the second connection supporting point 91 is located at the bottom position of the side wall 49 close to the forefoot side, because in the former state, the spring provides a pushing force, i.e., the spring is in a compressed state; and in the latter state, the spring provides a pulling force, and the spring is in a stretched state. Since the second revolute 40 needs to rotate, with the rotating movement, the spring is continuously subjected to stretching or retracting, the spring in the compressed state has better telescopic elongation property than the spring in the stretched state, because with the same material and the same process, the spring in the compressed state has a longer length in the natural state than that in the contracted state, therefore, during movement, the spring in a compressed state has better elongation and retracting properties, and is not easy to break after long-term operation, which greatly prolongs the service lifetime of the joint portion. Optionally, when the lower end of the spring 45 in the second revolute 40 is inclined towards the forefoot position, when the leg portion 41 is in a vertical state, the connecting rod 92 connected to the leg connecting portion 43 is shifted towards the forefoot side, which allows a longer spring to be accommodated between the first connection supporting point 90 and the second connection supporting point 91, and the longer spring also has better elongation and contraction performances. Correspondingly, when the lower end of the spring 45 in the second revolute 40 is inclined towards the heel position, when the leg portion 41 is in a vertical state, the connecting rod 92 connected to the leg connecting portion 43 is shifted towards the heel side.

Preferably, a mechanical ankle joint of the present embodiment further includes a cover plate 93, an edge of the cover plate 93 is matched with the side walls 49 of the foot connecting portion 44, the cover plate 93 can be assembled with the foot connecting portion 44, and after the assembly is completed, the rotating fit connecting portion of the leg connecting portion 43 and the first connection supporting point 90, the first elastic element, and the second connection supporting point 91 can be wrapped inside, so that parts in the second revolute 40 are not easy to fall off, and the operation of the second revolute 40 is safe and reliable. The first elastic element is not prone to corrosion (rustiness), thus greatly prolonging the service lifetime.

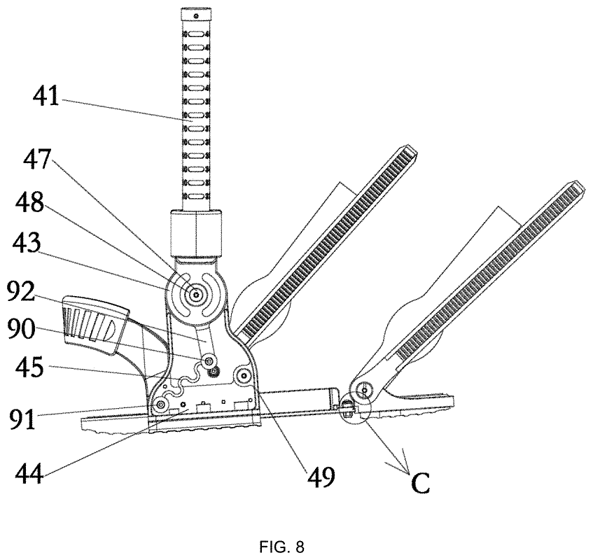

When the mechanical ankle joint of the present embodiment is not worn, the foot connecting portion 44 or the foot portion 42 is connected to an entire bottom surface on which the patient's foot is stepped, the forefoot position of the bottom surface is properly turned up with respect to the heel position (as shown in FIG. 8) or the forefoot position remains horizontal with respect to the heel position, the height of the forefoot position in the upturned state is slightly higher than that of the heel position, and the height of the forefoot position in the horizontal state is almost consistent with that of the heel position; when the mechanical foot is in use, the patient's foot is stepped on the bottom surface and the bottom surface is attached to the ground, which is an initial state, and the mechanical ankle joint does not transmit any force or hardly transmits any force to the patient. When the patient's leg is lifted, his/her foot drops around the ankle joint under its own gravity, and during the drop, the bottom surface attached to the patient's foot rotates together, the bottom surface makes, through the foot connecting portion 44, the second revolute 40 rotate, the first elastic element in the second revolute 40 is deformed relative to the initial state, and at this time, the first elastic element has a tendency to restore to the initial state, thus providing a reverse torque to the second revolute 40 for lifting the foot, which torque can be reflected as an upturning force transmitted to the bottom surface on which the foot is stepped, this force enables the dropping foot to be held, thus preventing the patient's foot from dropping; meanwhile, when the ankle joint rotates, the first elastic element can also decelerate the rotation process of the ankle joint, so that the movement speed is slowed down and the foot is prevented from being hurt.

In optional Embodiment 2, a shoe with adjustable wearing size is provided, including a first portion 50 and a second portion 51, wherein the first portion 50 and the second portion 51 constitute the bottom surface on which the foot is stepped in Embodiment 1, and the patient's foot can be stepped on the first portion 50 and the second portion 51. Since different patients have different foot sizes, in order to enhance the fit ability (adaptation ability) of the shoe to the patients, the first portion 50 and the second portion 51 are movably connected with each other, and by adjusting relative positions of the movable first portion 50 and second portion 51, the length of the adjusted bottom surface on which the foot is stepped is matched with the patient's foot. Specifically, a slidable connecting plate 52 is provided between the first portion 50 and the second portion 51, the connecting plate 52 is connected to one of the first portion 50 and the second portion 51 and embedded into the other. The connecting plate 52 is further provided thereon with a second locking structure 53, and the second locking structure 53 can achieve that the first portion 50 and the second portion 51 are locked in position after the adjustment and no longer continue to slide.

In the present embodiment, the connecting plate 52 and the second portion 51 are connected together, the connecting plate 52 is further provided thereon with the second locking structure 53, the second locking structure 53 includes a locking strip 54 arranged on the connecting plate 52, and the locking strip 54 is provided with locking holes 55. The first portion 50 includes an upper plate 94 and a lower plate 95, the upper plate 94 and the lower plate 95 can be assembled together. After the assembly is completed, a sliding groove 96 is formed on the first portion 50, and both the connecting plate 52 and the locking strip 54 part thereon can slide in the sliding groove 96, realizing the movable connection between the first portion 50 and the second portion 51. The second locking structure 53 further includes a push button 56, the push button 56 is arranged on the first portion 50, and after the positions of the first portion 50 and the second portion 51 are adjusted, by pressing the push button 56 into the locking hole 55, the position locking between the first portion 50 and the second portion 51 is completed.

Preferably, in order to facilitate the manipulation of the second locking structure 53 to make the same complete the locking operation, the push button 56 is provided on a side surface of the second portion 51, and correspondingly, the locking strip 54 is vertically arranged, so that the push button 56 can be inserted into the locking hole 55. Optionally, the locking strip 54 is arranged perpendicular to the connecting plate 52, the locking strip 54 is arranged at a side position of the connecting plate 52, the connecting plate 52 and the locking strip 54 thereon are overall L-shaped, and correspondingly, the sliding groove 96 is also L-shaped.

Preferably, in order to facilitate the adjustment of the relative positions of the first portion 50 and the second portion 51, a size mark is provided on the connecting plate 52, to facilitate the adjustment of the size of the shoe by an operator.

Preferably, in order to improve the integrity of the shoe, although the first portion 50 and the second portion 51 are movably connected with each other, such movable connection is inseparable, that is, the first portion 50 and the second portion 51 cannot be completely separated from each other, and cannot become two unconnected components. In order to achieve this purpose, chute(s) 97 is provided on the connecting plate 52, the chute 97 penetrates from top to bottom, with four walls being annular, and limiting block(s) 98 capable of being embedded into the chute(s) 97 is provided in the sliding groove 96. Specifically, two limiting blocks 98 are provided and both are provided on a bottom surface of the upper plate 94, and correspondingly, there are two chutes 97 on the connecting plate 52. When the upper plate 94 and the lower plate 95 are installed, the connecting plate 52 is sandwiched by the upper plate 94 and the lower plate 95, and the limiting blocks 98 are embedded in the chutes 97.

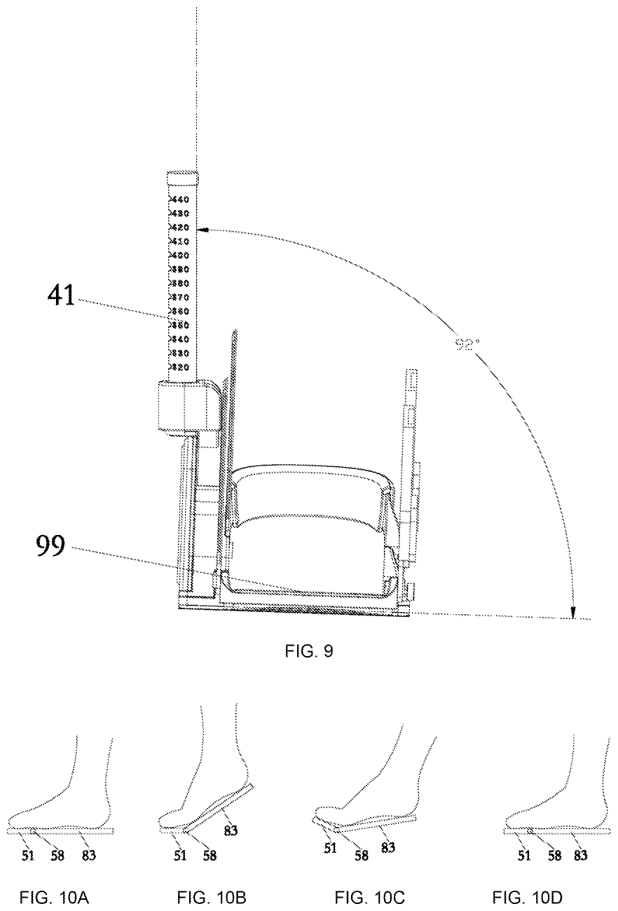

Referring to FIG. 9, in optional Embodiment 3, a conventional mechanical foot has the leg portion 41 and a stepping bottom surface 99. The leg portion 41 is configured to be connected to the patient's leg, and the stepping bottom surface 99 is configured to be attached to the patient's foot sole. In use, the leg portion 41 is fixed to the patient's leg, and the patient's foot is stepped on the stepping bottom surface 99, but in the conventional mechanical foot, an axis of the leg portion 41 is perpendicular to a plane where the stepping bottom surface 99 is located, which makes the patient have the feel of foot turning inward when wearing the mechanical foot, and causes discomfort. The present embodiment provides a mechanical foot, also including the leg portion 41 and the stepping bottom surface 99, wherein the axis of the leg portion 41 is not perpendicular to the plane where the stepping bottom surface 99 is located, which complies with ergonomic design, and makes the patient more comfortable when wearing the mechanical foot. Specifically, an angle formed by the leg portion 41 and the stepping bottom surface 99 is defined as the maximum angle among angles formed by the axis of the leg portion 41 and any straight line in the plane where the stepping bottom surface 99 is located, for example, if the angle formed by the axis of the leg portion 41 and any straight line in the plane where the stepping bottom surface 99 is located is in the range of [88°, 92°], the angle formed by the leg portion 41 and the stepping bottom surface 99 is 92°; for another example, if the angle formed by the axis of the leg portion 41 and any straight line in the plane where the stepping bottom surface 99 is located is in the range of [85°, 95°], the angle formed by the leg portion 41 and the stepping bottom surface 99 is 95°. In the present embodiment, the angle formed by the leg portion 41 and the stepping bottom surface 99 is 92°.

In optional Embodiment 4, a mechanical foot is provided. The mechanical foot includes a portion for receiving a forefoot and a portion for receiving a forefoot rear side, wherein the portion for receiving the forefoot and the portion for receiving the forefoot rear side have an included angle therebetween. This included angle may be an obtuse angle or a flat angle, for example, the portion for receiving the forefoot is at an acute angle relative to a horizontal position, for instance, an angle of 10, 20, 25, 30, 35, 40, 55 degrees, and at an obtuse angle relative to the portion for receiving the forefoot rear side located substantially in a horizontal plane, for instance, an angle of 170, 160, 155, 160, 165, 135 degrees, etc.

In some modes, such angle can be adjusted. “Adjust” herein mainly refers to that the angle can be adjusted due to different physiological structures of the human body and walking habits. Once adjusted, the included angle between the portion for receiving the forefoot rear side and the portion for receiving the forefoot is substantially kept at a fixed angle. Hence, in some modes, the angle also may be fixed and unadjustable. Therefore, the angle herein includes two aspects: the angle being fixed and unadjustable; and the angle being able to be adjusted to adapt to different patients and different walking habits, or to adjust the angle according to different requirements of the rehabilitation stage for the same patient.

In some modes, the structure for making the portion for receiving the forefoot rear side and the portion for receiving the forefoot have an included angle therebetween may be varied, and may be realized by any structure, for example, a damping structure, a spring structure, or any other suitable structure. In a relatively extreme implementation example, for instance, a damping structure is not included, but the forefoot itself of the mechanical foot is fixedly connected to the forefoot rear side at a certain angle, for example, connected by a suitable mechanical structure, such as a hinge structure.

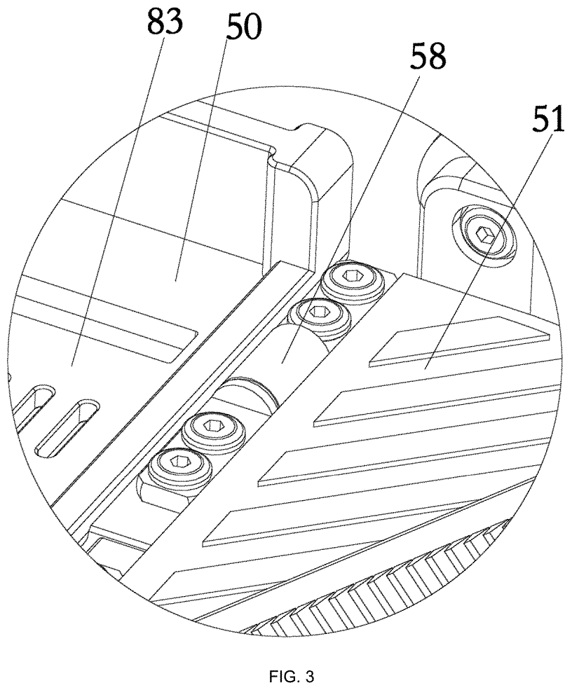

In some specific modes, with reference to FIG. 1, FIG. 3, and FIG. 4, the mechanical foot includes a third portion 83 and a second portion 51, the third portion 83 being connected to the second portion 51 in a rotation fit manner. In some modes, the manner of rotation fit connection is specifically a rotation fit connection having damping. Rotation herein actually can be understood as that the damping structure can adjust angles of the third portion 83 and the second portion 51 or an included angle therebetween. It will be appreciated that the damping structure also may be non-rotational damping.

It will also be appreciated that the third portion 83 and the second portion 51 of the mechanical foot are movably connected with each other. Such movable connection is not a natural movable connection, but instead there is a damping structure between the two portions, and the damping structure makes the two portions to be connected together. In some modes, the damping structure is provided between the forefoot and the rearfoot of the foot. A typical shoe is divided into a rearfoot with a heel and a forefoot with toes, between which is a portion of arch. This description is merely a general description for facilitating the following description. The damping structure herein can be any elastic or non-elastic element which can make them located in certain relative positions, for example, making the forefoot and the rearfoot located in fixed positions relative to each other. At such fixed positions, an external force is applied to the mechanical foot. For example, the mechanical foot is provided thereon with a damping structure, and when a person puts on or wears the mechanical foot, the mechanical foot has the second portion for receiving the forefoot of the person's foot and the third portion 83 for receiving the rearfoot, so that the person's foot and the mechanical foot are attached together. The mechanical foot of the present disclosure is worn by those people whose foot cannot be used normally for walking and is essentially different from normal healthy foot, for example, the diseased foot cannot walk normally, particularly, the foot is in a paralyzed state, and is completely or not completely controlled by the brain. In this way, during walking, the walking of the diseased foot needs to be assisted by the mechanical foot, and such walking has a rehabilitation effect.

In general, for the walking of a healthy foot, usually, the heel is lifted from the ground first and drives the forefoot to be lifted from the ground, and then the forefoot contacts the ground first, and then the rearfoot contacts the ground, thus completing the walking process. When the foot is a diseased foot, since the forefoot and the rearfoot are not or not completely controlled by the brain in walking, when the shank is lifted for walking, the whole sole is in a natural drop state. When walking on foot, the movement does not follow a motion track of healthy foot, in this case, it is easy to cause fall-down, for example, in the case that the diseased foot is in the natural drop state, when walking forward, it may be the parts of the toes that touch the ground first, but in the previous process, it is the movement of the ankle joint that drives the movement of the foot, then due to the ineffective coordination, it is easy to cause the person to tumble. In this case, it is desirable to have an angle between the forefoot and the rearfoot, and this angle may be an obtuse angle, for example, when the sole is in a horizontal plane, the forefoot forms an obtuse angle with the horizontal plane, which is similar to a form in which the forefoot is turned up relative to the horizontal plane. When the patient wears the mechanical foot on foot, the forefoot naturally has a force, such as pressure, applied to the forefoot of the mechanical foot, and in order to overcome this force, the damping element still keeps the angle between the forefoot and the rearfoot of the mechanical foot relatively unchanged or stable, so as to make the forefoot and the rearfoot of the patient also keep an angle, then in the walking of the diseased foot, the forefoot can be kept on the ground, thus it is not easy to tumble. In particular, for a hemiparalyzed person, one half of the patient, including the whole lower limbs, is in a paralyzed state, then when performing the rehabilitation training, the patient needs to walk with assistance of the mechanical foot, thus it is relatively important to prevent tumble. The primary function of such damping is to make the patient's forefoot and the forefoot rear side of the foot present a dorsiflexion angle. Of course, there is actually a structural arrangement that maintains an angle, such as the dorsiflexion angle, between the forefoot and the rearfoot of the mechanical foot, and such dorsiflexion angle almost remains unchanged when the patient's foot wears the mechanical foot. On the other hand, the angle can be adjusted, due to the existence of the damping structure. As the foot size and walking habit of each person are different, therefore, the dorsiflexion angle can be arbitrarily adjusted, to satisfy walking habits of each patient. Such an angle can be arbitrarily adjusted between 175 degrees and 85 degrees, and can be adjusted to 120, 135, and 110 degrees. Hence, due to the existence of the damping, once adjusted, a relatively fixed angle is maintained between the forefoot and the rearfoot of the mechanical foot. In addition to the damping structure, other manners may also be feasible, but in this implementation mode, the dorsiflexion angle is easy to fix, while this is not conducive to arbitrary adjustment. For example, a spring, an elastic sheet. Alternatively, more simply, an angle of dorsiflexion is made to be present between the forefoot and the rearfoot of the mechanical foot, but such an angle generally cannot be adjusted arbitrarily.

In some more specific embodiments, specifically, the second portion 51 is corresponding to a forefoot position, the third portion 83 is corresponding to a forefoot rear-side position, the two parts are cooperatively connected at a junction through a damping rotating shaft 58, and an elastic element is provided inside the damping rotating shaft 58. When the patient's diseased foot is stepped on the ground, the gravity of the forefoot is exerted on the second portion 51, so that the third portion 83 and the second portion 51 always have an angle. By “always” herein, it does not mean “all the time”, but the forefoot and the rearfoot of the patient's foot are made to substantially have an angle, for example, an angle at which the forefoot is away from the ground and turned up, such angle remains in a stable state, the maintenance of this angle is achieved by relying on the angle between the forefoot and the rearfoot of the mechanical foot, and the angle between the forefoot and the rearfoot of the mechanical foot can be realized through the damping structure.

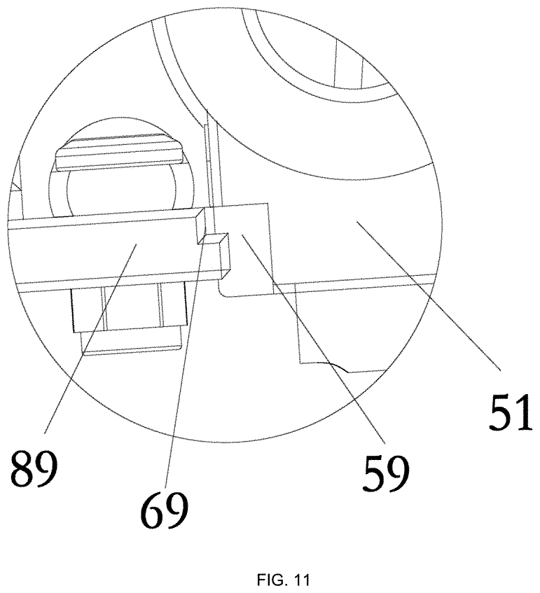

Specifically, the mechanical foot includes the third portion 83 and the second portion 51, when wearing the mechanical foot, the patient steps on the third portion 83 and the second portion 51, the third portion 83 and the second portion are movably connected with each other. The movable connection divides the surface on which the patient steps into two parts, and the movable connection includes rotation fit connection and elastic connection, and also includes a combination of the rotation fit connection and the elastic connection. With reference to FIG. 1, FIG. 3, and FIG. 4, the movable connection in the present embodiment is a special rotation fit connection; the third portion 83 and the second portion 51 are in rotation fit connection, in particular rotation fit connection with damping. Specifically, the second portion 51 is corresponding to the forefoot position, and the third portion 83 is corresponding to the forefoot rear-side position, and the two portions are in rotation fit connection at the junction through the damping rotating shaft 58. With reference to FIGS. 10A-D, when the patient's diseased foot is stepped on the ground, as shown in FIG. 10A, the gravity of the forefoot acts on the second portion 51, and the gravity at the forefoot rear-side position acts on the third portion 83, so that the third portion 83 is flush with the second portion 51; when the patient lifts the diseased foot, as shown in FIG. 10B, the heel of the patient is lifted from the ground first, the forefoot of the foot is lifted from the ground later, and the forefoot of the patient's foot and the forefoot rear-side portion of the foot present a dorsiflexion angle. Since the second portion 51 and the third portion 83 are attached to the bottom of the patient's foot, this process makes the third portion 83 and the second portion 51 rotate relative to each other; when the patient's diseased foot takes a step, as shown in FIG. 100, as the patient's diseased foot is in a suspending state at this time, the gravity of the patient's foot acts on the patient's leg, including the gravity of the patient's forefoot, and rarely falls on the second portion 51, the damping rotating shaft 58 at this time enables the second portion 51 to maintain a dorsiflexion state, i.e., makes the second portion 51 (forefoot) no longer drop, and the problem of easy tripping in the walking process is solved; when the patient's diseased foot is stepped on the ground again, as shown in FIG. 10D, the gravity of the foot is no longer borne by the patient's leg, instead, it acts on the second portion 51 and the third portion 83, and the damping rotating shaft 58 part between the third portion 83 and the second portion 51 is under action of the gravity of the patient's foot, and the third portion 83 and the second portion 51 rotate relative to each other again, so that the third portion 83 is flush with the second portion 51, and the initial state is restored.

Specifically, the third portion 83 and the second portion 51 are connected through the damping rotating shaft 58, first mounting station(s) 89 is provided on the third portion 83, second mounting station(s) 88 is provided on the second portion 51, and the first mounting station 89 and the second mounting station 88 are both provided with first connecting hole(s) for allowing the damping rotating shaft 58 to be mounted. The damping rotating shaft 58 includes a first shaft body 86 and a second shaft body 87, the first shaft body 86 and the second shaft body 87 each include a square segment and a cylindrical segment, the cylindrical segments of the two are nested together, so that the first shaft body 86 and the second shaft body 87 can rotate relative to each other. An elastic sheet and a washer are further provided in the damping rotating shaft 58, and when there is a tendency of relative rotation between the first shaft body 86 and the second shaft body 87, the elastic sheet and the washer rub against each other to generate a damping force. When a torque between the first shaft body 86 and the second shaft body 87 is greater than the maximum damping force, in this case, relative rotation occurs between the first shaft body 86 and the second shaft body 87, and the first shaft body 86 and the second shaft body 87 after the rotation can be maintained to be fixed relative to each other when there is no external force, or when the generated torque is less than the maximum damping force. The square segment of the first shaft body 86 and the square segment of the second shaft body 87 are each provided with second connecting hole(s) corresponding to the first connecting hole(s), and the mounting of the damping rotating shaft 58 between the third portion 83 and the second portion 51 can be completed by passing a screw through the first connecting hole and the second connecting hole. Preferably, two second connecting holes are provided on the square segment, two first connecting holes are provided on each of the first mounting station 89 and the second mounting station 88, two first mounting stations 89 and two second mounting stations 88 are respectively provided on the third portion 83 and the second portion 51, and two damping rotating shafts 58 are mounted between the third portion 83 and the second portion 51. When the patient's diseased foot is lifted up, a suitable force for turning the forefoot up can be provided, so that the forefoot will not drop anymore.

Preferably, a limiting structure is provided at a connecting portion of the third portion 83 and the second portion 51, that is, a limiting structure is provided at a connecting position of the forefoot portion and the forefoot rear-side portion of the foot, so that the damping rotating shaft 58 will not be pressed down too much by the gravity, thus avoiding the occurrence of the situation of plantarflexion caused by excessive rotation (turn-down). Specifically, referring to FIG. 11, a first groove 59 is provided on the second portion 51 at a position close to the first mounting station 89, and the first mounting station 89 partially extends into the first groove 59, so that when the second portion 51 is turned down, the first mounting station 89 abuts against the first groove 59, thus avoiding the turn-down. Optionally, in order to enable the second portion 51 to turn down appropriately and satisfy the requirement of the patient for plantarflexion sometimes, a second groove 69 is provided at one side of the first mounting station 89 close to the first groove 59.

The third portion 83 may be an integral part or a component formed by assembling two or more parts. When the third portion 83 is an integral part, as in FIGS. 10A-D, the third portion 83 is always attached to the forefoot rear-side portion and is unadjustable. When the third portion 83 is a component formed by assembling two or more parts, for example, as in Embodiment 2, the third portion 83 includes the first portion 50 and the connecting plate 52, the connecting plate 52 can slide in the first portion 50, and the mechanical foot at this time has the advantages of being adjustable in size and automatically turning up the forefoot during walking.

In optional Embodiment 5, a mechanical foot is provided, including a third portion 83 and a second portion 51, when wearing the mechanical foot, the patient steps on the third portion 83 and the second portion 51, the third portion 83 and the second portion are movably connected with each other. The movable connection divides the surface on which the patient steps into two parts, and the movable connection includes rotation fit connection and elastic connection, and also includes a combination of the rotation fit connection and the elastic connection. The movable connection in the present embodiment is an elastic rotation fit connection: the second portion 51 is corresponding to a forefoot position, and the third portion 83 is corresponding to a forefoot rear-side position, the two are connected in a rotation fit manner through an elastic rotating shaft at a junction, a second elastic element is provided inside the elastic rotating shaft, and the second elastic element can be parts such as a coil spring and a torsional spring which can enable the elastic rotating shaft to recover deformation after rotation. When the mechanical foot is not worn, the second portion 51 is appropriately turned up under the action of the second elastic element; when the patient's diseased foot is stepped on the ground, the gravity of the forefoot acts on the second portion 51, making the third portion 83 flush with the second portion 51; when the patient's diseased foot is lifted up, i.e., in a walking state, as almost all the gravity of the forefoot acts on the patient's leg at this time, the gravity of the forefoot no longer acts on the second portion 51, and the second portion 51 slowly turns up under the acting force of the second elastic element in the elastic rotating shaft, driving the forefoot portion to bend upwards, that is, making the second portion 51 (forefoot) no longer drop, and during the walking, solving the problem of easy tripping.

Preferably, the structure of the elastic rotating shaft is similar to that of the damping rotating shaft 58 in Embodiment 4, the elastic rotating shaft includes a third shaft body and a fourth shaft body, the third shaft body and the fourth shaft body each include a square segment and a cylindrical segment, and the cylindrical segments of the two are nested together, so that the third shaft body and the fourth shaft body can rotate relative to each other. Different from the damping rotating shaft 58, a second elastic element is provided between the third shaft body and the fourth shaft body, and the second elastic element is provided at a nested position of the cylindrical segments of the third shaft body and the fourth shaft body, so that the third shaft body and the fourth shaft body have the capability of recovering deformation after rotating relative to each other. The mounting position and the mounting manner of the elastic rotating shaft can be consistent with those of the damping rotating shaft 58 in Embodiment 4, and will not be redundantly repeated herein. Preferably, the third portion 83 and the second portion 51 are connected through two elastic rotating shafts, and there are in total two second elastic elements in the two elastic rotating shafts. When the patient's diseased foot is lifted up, a suitable force for turning the forefoot up can be provided, so that the forefoot will not drop anymore.

In optional Embodiment 6, a mechanical foot is provided, including a third portion 83 and a second portion 51, when wearing the mechanical foot, the patient steps on the third portion 83 and the second portion 51, the third portion 83 and the second portion are movably connected with each other. The movable connection divides the surface on which the patient steps into two parts, and the movable connection includes rotation fit connection and elastic connection, and also includes a combination of the rotation fit connection and the elastic connection. The movable connection in the present embodiment is an elastic connection; the second portion 51 is corresponding to a forefoot position, and the third portion 83 is corresponding to a forefoot rear-side position, the two are connected through an elastic sheet at a junction. The elastic sheet has a curvature such that one end thereof turns up, or the elastic sheet is provided with an upward crease, so that when the mechanical foot is not worn, the second portion 51 thereof can turn up appropriately; when the patient's diseased foot is stepped on the ground, the gravity of the forefoot acts on the second portion 51, making the third portion 83 flush with the second portion 51; when the patient's diseased foot is lifted up, i.e., in a walking state, as almost all the gravity of the forefoot acts on the patient's leg at this time, the gravity of the forefoot no longer acts on the second portion 51, and the second portion 51 slowly turns up under the acting force of an elastic sheet, driving the forefoot portion to bend upwards, that is, making the second portion 51 (forefoot) no longer drop, and solving the problem of easy tripping during the walking.

In optional Embodiment 7, a mechanical foot is provided, which is used to be worn on a patient's foot, and includes a stepping bottom surface 99, and an ankle fixing strap 82 and a back fixing strap 85 both provided on the stepping bottom surface 99, wherein the ankle fixing strap 82 is configured to fix a rear-side portion of the patient's foot, and the back fixing strap 85 is configured to fix an instep portion (foot dorsum, dorsum pedis) of the patient's foot. By fixing the two portions, the mechanical foot can be worn on the patient's foot.

Preferably, when putting on the mechanical foot, the rear-side portion of the ankle joint of the patient is preferentially made to abut against the ankle fixing strap 82, and then the back fixing strap 85 is fixed, so that the patient's foot is attached to the stepping bottom surface 99. When putting on the mechanical foot, the patient may put on the mechanical foot in a state of wearing a shoe, or may put on the mechanical foot without wearing a shoe.

Since the overall length of the instep portion is relatively long, a single back fixing strap 85 cannot achieve a good fixing effect, and preferably, two back fixing straps 85 are provided for better fixing the foot. Optionally, the back fixing strap 85 includes a shoe toothed strap 79 and a shoe buckle 78, and a quick connection of the instep portion can be completed by passing the shoe toothed strap 79 through the shoe buckle 78, i.e., to complete quick wearing of the mechanical foot, meanwhile, the shoe buckle 78 is provided with a button 77, and by pressing the button 77, quick unlocking of the shoe toothed strap 79 and the shoe buckle 78 can be realized, facilitating the removal of the mechanical foot. The shoe toothed belt 79 and the shoe buckle 78 are connected to connection positions of the mechanical foot in a rotation fit manner, so that the wearing angles of them can all be adjustable, and the mechanical foot is adapted to be worn by different people.

Preferably, a flexible pad 84 is provided on the mechanical foot, and the flexible pad 84 can be provided on an upper surface of the stepping bottom surface 99, which can improve comfort when the patient wears the mechanical foot. The flexible pad 84 can also be provided on a lower surface of the stepping bottom surface 99, which can reduce noise when the mechanical foot is stepped on the ground, meanwhile also has the effect of protecting the mechanical foot, and most importantly, can prevent slippage, avoid the patient from falling when wearing it, and have the effect of protecting the patient.

In optional Embodiment 8, a mechanical foot is provided, and a technical solution adopted thereby is any combination of Embodiment 1-Embodiment 7 in the above, wherein any combination includes a combination of two embodiments, a combination of three embodiments, etc.

For example, the combination of Embodiment 1 and Embodiment 4 can realize that the forefoot does not drop on the basis that the ankle joint does not drop, thus preventing the patient from tumbling when walking.

For example, for the combination of Embodiment 2 and Embodiment 4, a structure capable of adjusting the length is further provided between the forefoot and the rearfoot, wherein this structure has one end connected to the forefoot, and the other end connected to the rearfoot portion. By means of the structure capable of adjusting the length, the distance between the forefoot and the rearfoot can be adjusted, so that the mechanical foot can be adapted to foot sizes of different patients. It should be noted that “foot” and “sole” herein are interchangeable, and both indicate the patient's foot or the mechanical foot, or a mechanical sole or the mechanical foot suitable to be worn by the patient, and may also be referred to as a mechanical shoe. The shoe includes a structure of a mechanical sole, and the mechanical shoe, the mechanical foot or the mechanical sole is configured to be worn on the patient's foot or sole. The damping structure is provided on an adjusting structure for adjusting the distance between the forefoot and the rearfoot, and the damping structure has one end connected to the structure for adjusting the distance, and the other end connected to the forefoot, thus fixing or adjustment of the angle between the forefoot and the rearfoot also can be realized. In this case, the adjusting structure may be a part of the rearfoot, and certainly, may also be a part of the forefoot.

To sum up, the above-mentioned are merely for specific embodiments of the present disclosure, but the scope of protection of the present disclosure is not limited thereto. Any variation or substitution conceived without any creative effort should be covered within the scope of protection of the present disclosure. Therefore, the scope of protection of the present disclosure should be based on the scope of protection defined by the claims.

### INDUSTRIAL APPLICABILITY

In conclusion, the present disclosure provides a mechanical foot. The mechanical foot can lift the diseased forefoot of the patient during the walking of the patient, so as to prevent dragging of the foot and some injuries caused by tripping or tumbling related to the dragging of the foot, and it will not generate an overlarge force on the ankle joint of the patient which causes discomfort, thus greatly reducing the patient's discomfort.

What is claimed is:

1. A mechanical foot, comprising a mechanical ankle joint,
wherein the mechanical ankle joint comprises a leg portion configured to be fixed to a patient's leg, a foot portion configured to be fixed to a patient's foot, and a revolute, the leg portion is connected with the foot portion through the revolute in a manner of being rotatable relative to each other, and
wherein the revolute comprises a foot connecting portion and a leg connecting portion, the foot connecting portion is connected to the foot portion, the leg connecting portion is connected to the leg portion, and the foot connecting portion is connected with the leg connecting portion in a manner of being rotatable relative to each other, and
the revolute further comprises a first elastic element, wherein one end of the first elastic element is connected to the foot connecting portion, and the other end of the first elastic element is connected to the leg connecting portion, so that a force for maintaining the patient's foot substantially horizontal is provided through the revolute at a position of the mechanical ankle joint.

2. The mechanical foot according to claim 1, wherein the leg connecting portion is provided with a hole, the foot connecting portion is provided with a shaft, and the shaft is configured in such a way of being provided in the hole in a rotatable manner.

3. The mechanical foot according to claim 1, wherein the leg connecting portion is provided with a first connection supporting point, the foot connecting portion is provided with a second connection supporting point, and the first connection supporting point is connected with the second connection supporting point through the first elastic element.

4. The mechanical foot according to claim 3, wherein the foot connecting portion are provided with side walls, and the side walls are configured to limit, when the leg connecting portion and the foot connecting portion rotate relative to each other, a movement position of the first connection supporting point.

5. The mechanical foot according to claim 3, wherein the leg connecting portion is provided thereon with a connecting rod, and the first connection supporting point is arranged at an end of the connecting rod away from the leg portion.

6. The mechanical foot according to claim 3, wherein one end of the first elastic element connected to the first connection supporting point is configured to rotate freely around a central axis of the first connection supporting point, and the other end of the first elastic element connected to the second connection supporting point is configured to rotate freely around a central axis of the second connection supporting point.

7. The mechanical foot according to claim 4, wherein the second connection supporting point is provided at a bottom position of the foot connecting portion close to a side wall of the side walls.

8. The mechanical foot according to claim 1, wherein the mechanical ankle joint further comprises a cover plate, and an edge of the cover plate is of a shape matched with shapes of the side walls of the foot connecting portion.

9. The mechanical foot according to claim 1, wherein the foot portion of the mechanical foot comprises a rear portion and a forefoot portion, the rear portion and the forefoot portion constitute a bottom portion configured to be attached to the patient's foot, the foot connecting portion is connected to the bottom portion, and the rear portion is movably connected to the forefoot portion.

10. The mechanical foot according to claim 1, wherein the foot portion of the mechanical foot comprises a forefoot rear-side portion and a forefoot portion, the forefoot rear-side portion is movably connected with the forefoot portion, the forefoot rear-side portion and the forefoot portion constitute a bottom portion configured to be attached to the patient's foot, the foot connecting portion is connected to the bottom portion, and a damping structure is provided at a junction between the forefoot rear-side portion and the forefoot portion, so as to make the forefoot rear-side portion angularly changed relative to the forefoot portion.

11. The mechanical foot according to claim 10, wherein the damping structure comprises a damping rotating shaft, the forefoot rear-side portion is provided with at least one first mounting station, the forefoot portion is provided with at least one second mounting station, and the at least one first mounting station and the at least one second mounting station are each provided with at least one first connecting hole configured for allowing the damping rotating shaft to be mounted.

12. The mechanical foot according to claim 11, wherein a limiting structure is provided at a connecting position of the forefoot rear-side portion and the forefoot portion, so as to avoid excessive rotation of the damping rotating shaft.

13. The mechanical foot according to claim 12, wherein the forefoot portion is provided with a first groove configured to face the at least one first mounting station, and the at least one first mounting station is configured in such a manner that when the forefoot portion rotates to an extreme position, the at least one first mounting station partially extends into the first groove, so as to abut against the first groove.

14. The mechanical foot according to claim 13, wherein a second groove is provided on a side portion of the at least one first mounting station facing the first groove, so that the forefoot portion can be inclined downwards to meet a plantarflexion requirement of a patient.

15. The mechanical foot according to claim 9, wherein the forefoot portion and the forefoot rear-side portion are connected with each other in a rotatable manner through an elastic rotating shaft, a second elastic element is provided inside the elastic rotating shaft, and
the second elastic element is configured in such a manner that when the mechanical foot is not worn or when a patient wears the mechanical foot and lifts the foot up, the forefoot portion forms an angle with the forefoot rear-side portion under an action of a biasing force of the second elastic element; and that when the patient wears the mechanical foot and steps on the ground with the foot, a force applied by the patient resists the biasing force applied by the second elastic element, so that the forefoot portion is flush with the forefoot rear-side portion.

16. The mechanical foot according to claim 9, wherein the leg portion and the bottom portion have an angle of 92°-95°.
